(19) United States
(12) Patent Application Publication
(10) Pub. No.: US 2026/0083951 A1
(43) Pub. Date: Mar. 26, 2026

(54) TUBING MANAGEMENT FOR AN IMPLANTABLE MEDICAL DEVICE

(71) Applicant: Boston Scientific Scimed, Inc., Maple Grove, MN (US)

(72) Inventors: John Anders Bostrom, Long Lake, MN (US); Trey Henry Achterhoff, Arden Hills, MN (US); Derek John Boettger, Woodbury, MN (US); Matthew William Couri, Plymouth, MN (US); Paul John Gindele, Buffalo, MN (US); Matthew Lee Nelson, Plymouth, MN (US)

(21) Appl. No.: 19/331,799

(22) Filed: Sep. 17, 2025

Related U.S. Application Data
(60) Provisional application No. 63/698,332, filed on Sep. 24, 2024.

(51) Int. Cl. A61M 39/10; A61M 5/142

(52) U.S. Cl. CPC A61M 39/10; A61M 5/14276; A61M 2039/1066

(57) ABSTRACT

An implantable medical device may include an inflatable member, a fluid reservoir, a pump device, a first tube member configured to fluidly connect the pump device and the fluid reservoir, a second tube member configured to fluidly connect the pump device and the inflatable member, and a tube retainer defined by or coupled to an external surface of the pump device. The tube retainer is configured to hold a portion of the first tube member or the second tube member.

### CROSS-REFERENCE TO RELATED APPLICATION

This application claims priority to U.S. Provisional Patent Application No. 63/698,332, filed on Sep. 24, 2024, entitled “TUBING MANAGEMENT FOR AN IMPLANTABLE MEDICAL DEVICE”, the disclosure of which is incorporated by reference herein in its entirety.

### TECHNICAL FIELD

This disclosure relates generally to an implantable medical device with one or more tube retainer members for tubing management.

### BACKGROUND

Tube members (e.g., kink resistant tubing (KRT)) may be used to route fluid between components of a medical device. Tube routing and management of excess tubing between the components may make a surgical implant procedure more complicated and time consuming.

### SUMMARY

In some aspects, the techniques described herein relate to an implantable medical device including: an inflatable member; a fluid reservoir; a pump device; a first tube member configured to fluidly connect the pump device and the fluid reservoir; a second tube member configured to fluidly connect the pump device and the inflatable member; and a tube retainer defined by or coupled to an external surface of the pump device, the tube retainer configured to hold a portion of the first tube member or the second tube member.

In some aspects, the techniques described herein relate to a method including: coupling a portion of a first tube member or a second tube member to a tube retainer defined by or coupled to an external surface of a pump device; and inserting at least a portion of a medical device into a body of a patient, the medical device including the pump device, the first tube member, the second tube member, an inflatable member, and a fluid reservoir.

In some aspects, the techniques described herein relate to an implantable medical device including: an inflatable member; a fluid reservoir; a pump device; a first tube member configured to fluidly connect the pump device and the fluid reservoir; a second tube member configured to fluidly connect the pump device and the inflatable member; and a tube retainer defined by or coupled to an external surface of the pump device, the tube retainer configured to hold a portion of the first tube member or the second tube member.

In some aspects, the techniques described herein relate to a method including: coupling a portion of a first tube member or a second tube member to a tube retainer defined by or coupled to an external surface of a pump device; and inserting at least a portion of a medical device into a body of a patient, the medical device including the pump device, the first tube member, the second tube member, an inflatable member, and a fluid reservoir.

In some aspects, the techniques described herein relate to an implantable medical device including: an inflatable member; a fluid reservoir; a pump device including a manifold, the manifold including a first fluid port and a second fluid port; a first tube member configured to connect to the first fluid port and to the fluid reservoir; a second tube member configured to fluidly connect to the second fluid port and the inflatable member; and a tube retainer defined by or coupled to an external surface of the pump device, the tube retainer configured to hold a portion of the first tube member or the second tube member.

### BRIEF DESCRIPTION OF THE DRAWINGS

FIG. 1 illustrates a medical device with one or more tube retainer members for tube management according to an aspect.

FIG. 2A illustrates an example of a retainer band with a tab defining a groove configured to hold a tube member according to an aspect.

FIG. 2B illustrates a pump device with multiple retainer bands disposed around an exterior surface of a pump housing according to an aspect.

FIG. 2C illustrates a pump device with multiple retainer bands disposed around an exterior surface of a pump housing according to another aspect.

FIG. 2D illustrates an example of fluid ports of a pump device coupled to tube members according to an aspect.

FIG. 2E illustrates an example of a retainer band with multiple tabs defining grooves configured to hold portions of tube members according to an aspect.

FIG. 2F illustrates an example of a retainer band with a tab defining a hole that receives a tube member according to an aspect.

FIG. 3A illustrates an example of an integrated retainer tab, extending from an external surface of a pump housing, that defines a groove configured to hold a portion of a tube member according to an aspect.

FIG. 3B illustrates a detailed version of the integrated retainer tab with the groove and suture holes according to an aspect.

FIG. 4 illustrates an example of an integrated retainer hook, extending from an external surface of a pump housing, configured to hold a portion of a tube member according to an aspect.

FIG. 5A illustrates an example of an integrated tab, extending from an external surface of a pump housing, that defines a hole configured to receive a tube member or a connector according to an aspect.

FIG. 5B illustrates an example of a connector for a tube member according to an aspect.

FIG. 5C illustrates an example of a connector for a tube member according to another aspect.

FIGS. 6A to 6D illustrate perspectives of a tube guide coupled to a manifold of a pump device according to an aspect.

FIGS. 7A to 7D illustrate perspectives of a tube guide coupled to a manifold of a pump device according to another aspect.

FIGS. 8A to 8D illustrate various aspects of a removable clip member coupled to a manifold of a pump device according to an aspect.

FIGS. 9A and 9B illustrate perspectives of a post retainer on an external surface of a manifold of a pump device according to an aspect.

FIGS. 10A and 10B illustrate perspectives of an inflatable penile prosthesis according to an aspect.

FIG. 11 illustrates an example of an artificial urinary sphincter device according to an aspect.

FIG. 12 illustrates a flowchart depicting example operations of using a tab retainer on a pump device of a medical device according to an aspect.

### DETAILED DESCRIPTION

This disclosure relates to a medical device with one or more tube retainers for tube management, where the tube retainers are coupled to, defined by, or extensions of a pump device. In some examples, the tube retainer(s) can be removably coupled to an external surface of the pump device. In some examples, the tube retainer(s) are surface features or extensions of a manifold of the pump device. In some examples, the tube retainer(s) are coupled to the pump device using a snap-fit. In some examples, the tube retainer(s) are coupled to a pump device using an adhesive material. The tube retainer(s) may be used to hold and/or guide one or more portions of one or more tube members (e.g., kink resistant tubing (KRT)) while the medical device is implanted in the body of a patient to accommodate differing surgical placements and varying anatomies. In some examples, the tube retainer(s) may control the tension of tubing interconnects, operate as a strain relief to limit the tubing's relative motion, and/or minimize abrasion of a tubing member on a pump device. The implantable medical device may include an inflatable member, a pump device, and a fluid reservoir. The medical device includes one or more tube members (also referred to as conduit connectors) connected between the inflatable member and the pump device, one or more tube members connected between the fluid reservoir and the pump device, and one or more connectors that are used to connect two tube members together. The tube members transfer fluid between the components of the medical device.

Tube retainer(s) may extend from, or is/are coupled to, a pump housing. In some examples, the pump device includes a single tube retainer. In some examples, the pump device includes multiple tube retainers. In some examples, the tube retainer(s) are formed on an exterior surface of a manifold of a pump device. In some examples, a manifold is a component of the pump assembly that distributes fluid and/or regulates pressure. In some examples, the manifold includes a first fluid port that connects a first tube member that is used to connect to a fluid reservoir, and a second fluid port that connects a second tube member that is used to connect to an inflatable member. A tube retainer may be a component, surface feature, and/or a structure configured to hold and/or guide a portion of the first tube member or the second tube member that extends from a first fluid port or a second fluid port. In some examples, the tube retainer may hold or retain a connector that can connect two tube members together. A tube retainer may be an interference fit groove, a hook, a ring, a slot, a hole, and/or a track. A tube retainer may include an open lumen or a closed lumen. A tube retainer may be machined, printed or welded on the pump device. In some examples, a tube retainer may be a removable clip (with one or more KRT constraints), a band (e.g., a removable flexible band), and/or a wrap. In some examples, a tube retainer may include routing features.

In some examples, the tube retainer(s) may include one or more features (e.g., migration prevention features) for migration prevention (e.g., reducing the migration of a component (e.g., pump device) within a body of a patient). A migration prevention feature may include a suture hole, a suture tab, a loop, a ring, a removable clip on a pump device, a band, a wrap on a pump device with one or more tiedown features, an anchoring mechanism (e.g., over-molded barbs), an over-mold with one or more tiedown features, a sleeve, and/or a coating. In some examples, the migration prevention features may be machined, printed, or welded on the tube retainer and/or the pump device. In some examples, the migration prevention features may reduce a migration of a pump device within a body of a patent, limit tubing movement, and/or accommodate different surgical placements and/or varying anatomies.

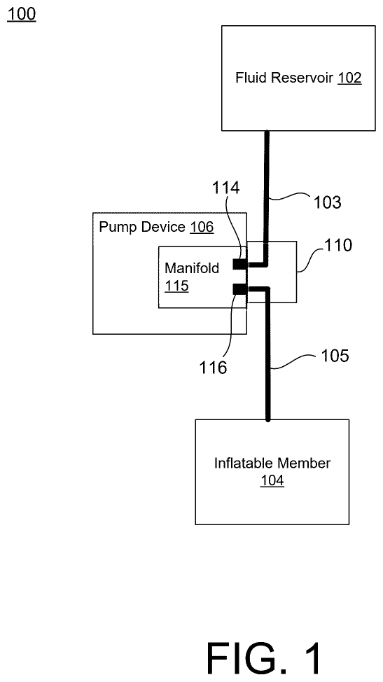

FIG. 1 illustrates an implantable medical device 100 according to an aspect. In some examples, the implantable medical device 100 is an artificial urinary sphincter device. In some examples, the implantable medical device 100 is an inflatable penile prosthesis. However, the implantable medical device 100 may include any type of medical device that transfers fluid between components of the implantable medical device 100.

The implantable medical device 100 with one or more tube retainers 110 for tube management, where the tube retainers 110 are coupled to, defined by, or extensions of a pump device 106. In some examples, the tube retainer(s) 110 can be removably coupled to an external surface of the pump device 106. In some examples, the tube retainer(s) 110 are surface features or extensions of a manifold 115 of the pump device 106. In some examples, the tube retainer(s) 110 are coupled to the manifold 115. In some examples, the tube retainer(s) 110 are coupled to the pump device 106 using a snap-fit. In some examples, the tube retainer(s) 110 are coupled to pump device 106 using an adhesive material. The tube retainer(s) 110 may be used to hold and/or guide one or more portions of one or more tube members (e.g., 112-1, 112-2) (e.g., kink resistant tubing (KRT)) while the implantable medical device 100 is implanted in the body of a patient to accommodate differing surgical placements and varying anatomies.

The implantable medical device 100 includes a fluid reservoir 102, an inflatable member 104, and a pump device 106 configured to transfer fluid between the fluid reservoir 102 and the inflatable member 104. In some examples, the inflatable member 104 is an inflatable cuff member configured to be implemented around a urethra of a patient. In some examples, the inflatable member 104 is a penile inflation member (e.g., one or more inflatable cylinders) that may be implanted into the corpus cavernosum of the user. The fluid reservoir 102 may be implanted in the abdomen or pelvic cavity of the user (e.g., the fluid reservoir 102 may be implanted in the lower portion of the user's abdominal cavity or the upper portion of the user's pelvic cavity). In some examples, at least a portion of the pump device 106 may be implemented in the patient's body.

The inflatable member 104 may be capable of expanding upon the injection of fluid into a cavity of the inflatable member 104. If implanted around the urethra, the expansion of the inflatable member 104 causes the urethra to become restricted, thereby reducing the risk of incontinence in patients. For example, the pump device 106 is configured to move fluid to pressure the inflatable cuff (e.g., the inflatable member 104), which constricts the urethra, thereby restricting the flow of urine. To urinate, the patient may operate the pump device 106 to depressurize the inflatable cuff by transferring fluid from the inflatable cuff to the fluid reservoir 102. If implanted into the corpus cavernosum, upon injection of the fluid into the inflatable member 104, the inflatable member 104 may increase its length and/or width, as well as increase its rigidity.

The fluid reservoir 102 may include a container having an internal chamber configured to hold or house fluid that is used to inflate the inflatable member 104. In some examples, the fluid reservoir 102 is pressurized. In some examples, the fluid reservoir 102 is a pressurized balloon. In some examples, the implantable device 100 includes a single pressurized balloon. In some examples, the implantable medical device 100 includes two or more pressurized balloons. The pressure in the inflatable member 104 may be generated by the fluid reservoir 102. For example, the fluid can be transferred to the inflatable member 104 without the use of a pump. The pressure differential between the inflatable member 104 and the fluid reservoir 102 will cause the fluid to flow from the fluid reservoir 102 to the inflatable member 104. In some examples, the pressure in the fluid reservoir 102 is greater than the pressure in the inflatable member 104 (e.g., even when the inflatable member 104 is at its target or maximum pressure). In some examples, the pressure in the fluid reservoir 102 is always greater than the pressure in the inflatable member 104.

The implantable device 100 may include a first tube member 103 and a second tube member 105. In some examples, the first tube member 103 and the second tube member 105 are referred to as conduit connectors. Each of the first tube member 103 and the second tube member 105 may define a lumen configured to transfer the fluid to and from the pump device 106. The first tube member 103 may be coupled to the pump device 106 and the fluid reservoir 102 such that fluid can be transferred between the pump device 106 and the fluid reservoir 102 via the first tube member 103. For example, the first tube member 103 may define a first lumen configured to transfer fluid between the pump device 106 and the fluid reservoir 102. The first tube member 103 may include a single or multiple tube members for transferring the fluid between the pump device 106 and the fluid reservoir 102. In some examples, the first tube member 103 may be referred to as first tube members, and two first tube members can be connected together using a connector.

The second tube member 105 may be coupled to the pump device 106 and the inflatable member 104 such that fluid can be transferred between the pump device 106 and the inflatable member 104 via the second tube member 105. For example, the second tube member 105 may define a second lumen configured to transfer fluid between the pump device 106 and the inflatable member 104. The second tube member 105 may include a single or multiple tube members for transferring the fluid between the pump device 106 and the inflatable member 104. In some examples, the second tube member 105 may be referred to as second tube members, and two second tube members can be connected together using a connector. In some examples, the first tube member 103 and the second tube member 105 may include a silicone rubber material. In some examples, the pump device 106 may be directly connected to the fluid reservoir 102.

The pump device 106 that can monitor control and regulate the pressure within an inflatable member 104. In some examples, the pump device 106 is an electronic pump device. In some examples, the pump device 106 is referred to as a can. The pump device 106 may automatically transfer fluid between the fluid reservoir 102 and the inflatable member 104 without the user manually operating a pump (e.g., squeezing and releasing a pump bulb). The pump device 106 includes a manifold 115. In some examples, a manifold 115 is a component of the pump device 106 that distributes fluid and/or regulates pressure. In some examples, the manifold 115 includes a first fluid port 114 that connects to the first tube member 103, and a second fluid port 116 that connects to the second tube member 105. The pump device 106 may include one or more pumps, valves, pressure sensor(s), a battery, and/or electronic circuitry. The pump device 106 may include an antenna configured to wirelessly transmit (and receive) wireless signals from an external device. The external device may be any type of component that can communicate with the pump device 106. The external device may be a computer, smartphone, tablet, pendant, key fob, etc. A user may use the external device to control the implantable medical device 100. In some examples, the user may use the external device to inflate or deflate the inflatable member 104.

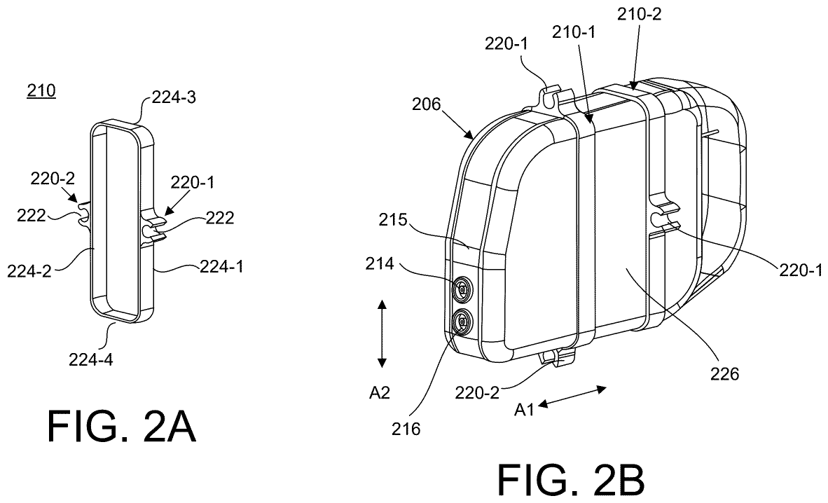

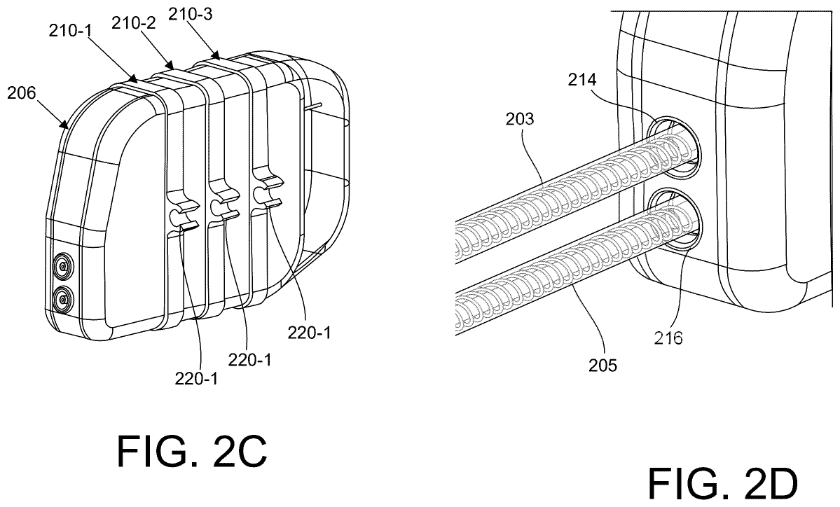

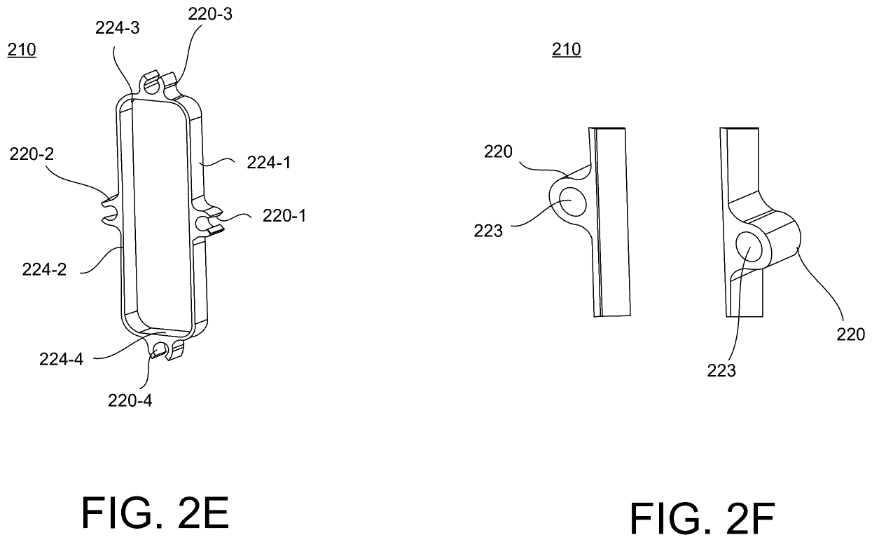

The tube retainer 110 may include one or more retainer bands (e.g., FIGS. 2A to 2F) coupled around an exterior surface of a pump housing. In some examples, the retainer band(s) are removable, where an operator may place one, two, or three retainer bands around the pump housing. In some examples, a retainer band is an elastic material (e.g., an clastic ring member) that can be stretched (e.g., length and/or width can be stretched). In some examples, a retainer band may be a relatively rigid plastic component. A retainer band may include two or more tabs at different positions around the retainer band, where each tab defines a groove (e.g., an open lumen) configured to hold a portion of a tube member 103 or 105. The size (e.g., diameter) of the groove may be slightly less than the size (e.g., diameter) of the tube member 103 or 105. In some examples, a tab of a retainer band may define a hole (e.g., a closed lumen) configured to receive the tube member 103 or 105. In some examples, a retainer band includes two tabs (each with a groove or a hole) positioned on opposite band portions. For example, the retainer band may have a rectangular shape with first and second (long) sides and third and fourth (short) sides. In some examples, the two tabs are disposed on the first and second (long) sides (e.g., opposite to each other). In some examples, the two tabs are disposed on the third and fourth (short) sides (e.g., opposite to each other). In some examples, a retainer band includes four tabs (e.g., one on each side, two tabs being disposed opposite to each).

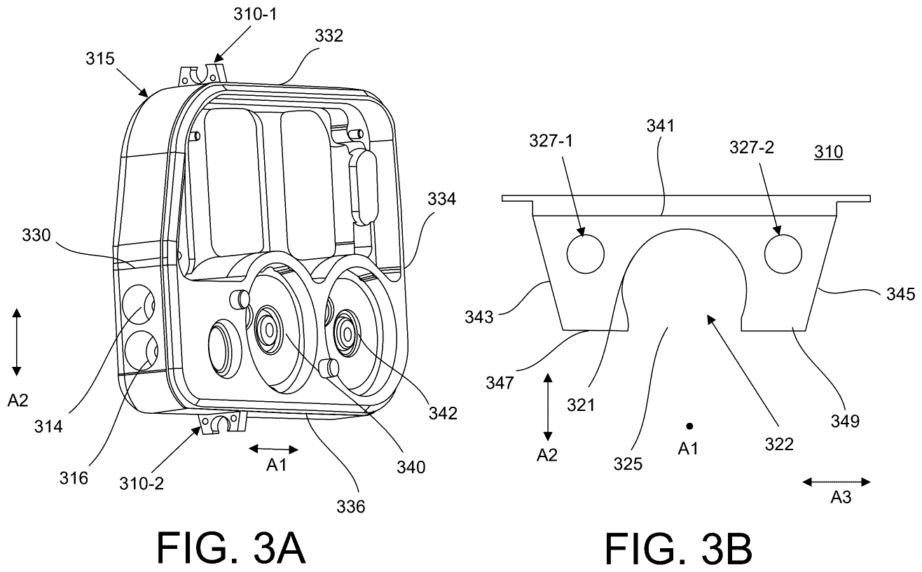

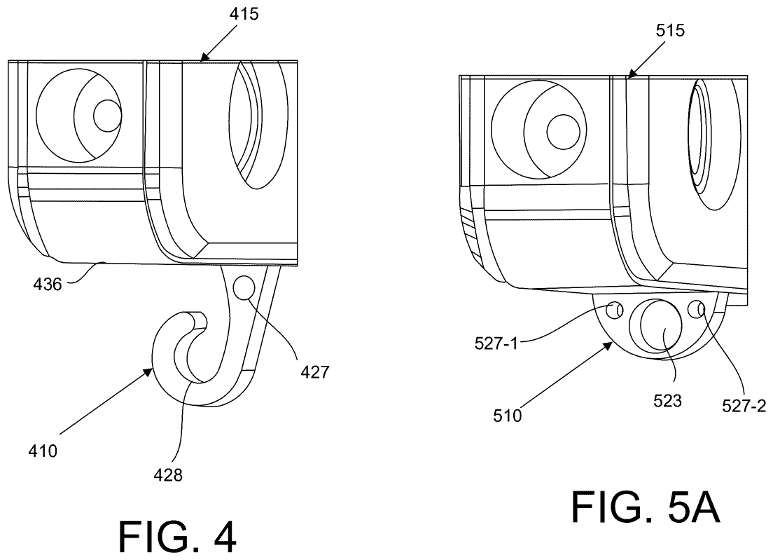

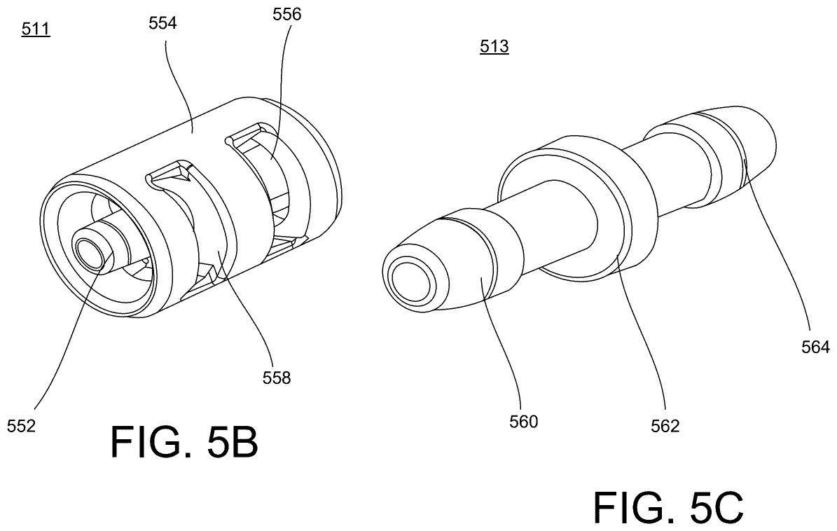

In some examples, the tube retainer 110 may include one or more integrated retainer tabs (e.g., see FIGS. 3A and 3B) extending from an external surface of a pump housing (e.g., an external sidewall of a manifold), where a retainer tab defines a groove (e.g., an open lumen) configured to receive a portion of a tube member 103 or 105. The groove may have an opening that is less than the outer diameter of a tube member, which may require a press fit action to seat the tube member in the groove. In some examples, the retainer tab includes a first suture hole and a second suture hole. The first and second suture holes may be used to attach sutures to the retainer tabs. In some examples, the groove is discussed between the first suture hole and the second suture hole. In some examples, a retainer tab (e.g., see FIG. 5A) defines a hole (e.g., a closed lumen) configured to receive a tube member 103 or 105 or a connector (e.g., see FIG. 5B or 5C) that is used to connect two tube members together. In some examples, the retainer tab defines a first suture hole and a second suture hole. In some examples, a tube retainer (e.g., see FIG. 4) includes a retainer hook. In some examples, the retainer hook includes a radius of curvature that is smaller than the outer diameter of a tube member. In some examples, the retainer hook includes one or more suture holes.

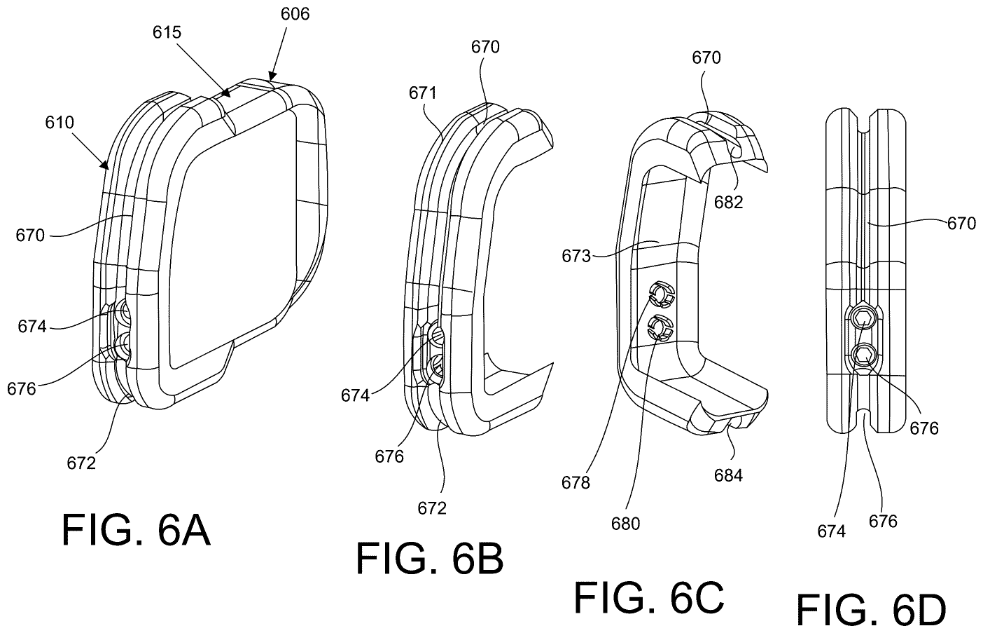

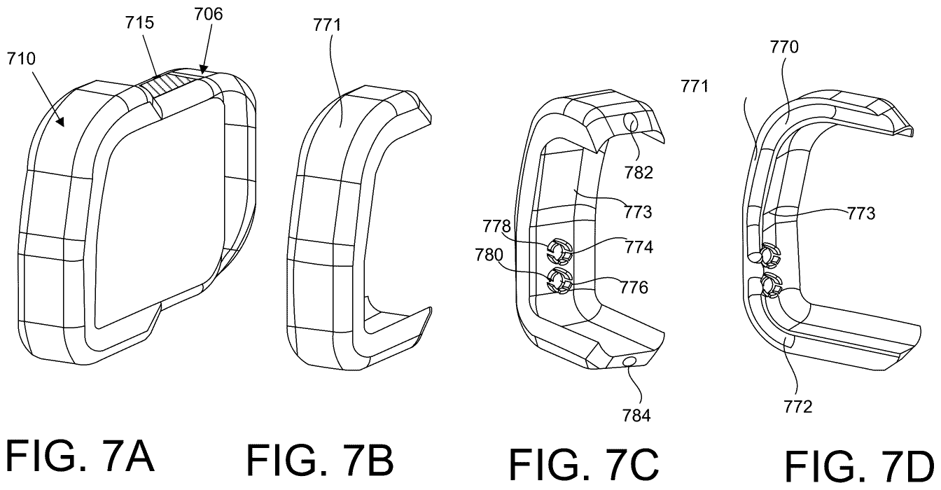

In some examples, the tube retainer 110 may include a tube guide (e.g., see FIGS. 6A to 6D or FIGS. 7A to 7D) that is coupled to a pump housing. In some examples, the tube guide is referred to as a clip member. In some examples, the tube guide has a C-shape. In some examples, the tube guide is coupled to the fluid ports (e.g., 114, 116) on the manifold 115 of the pump device 106. In some examples, the tube guide is coupled to the pump device 106 using a mechanical lock (e.g., a snap fit), an adhesive bond, or applied as an over-mold. The tube guide defines two channels, e.g., a first channel that extends from a first fluid port 114, and a second channel that extends from a second fluid port 116. First and second tube members (which extend from the first and second fluid ports 114, 116, respectively) are positioned in the first and second channels, respectively. The first and second channels may extend along the tube guide in opposite directions. In some examples, the first and second channels are external channels (e.g., see FIGS. 6A to 6D) that are defined on an external surface of the tube guide. In some examples, the first and second channels are internal channels (e.g., see FIGS. 7A to 7D) that are defined within the tube guide, and the tube guide defines a first opening at the end of the first internal channel and a second opening at the end of the second internal channel.

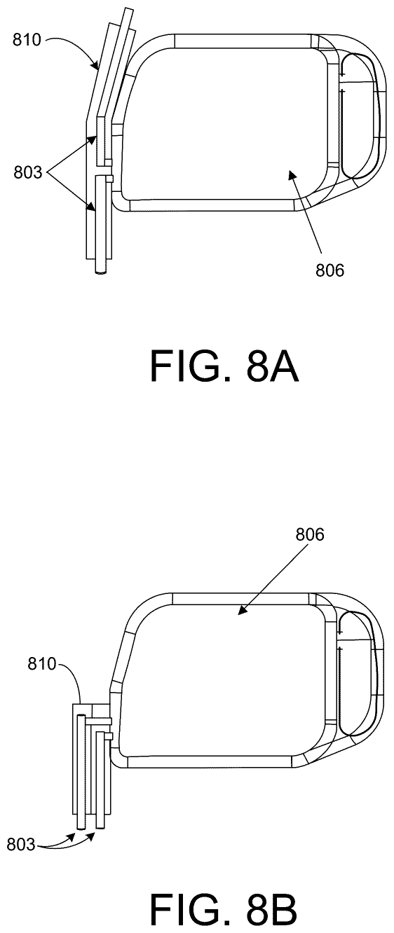

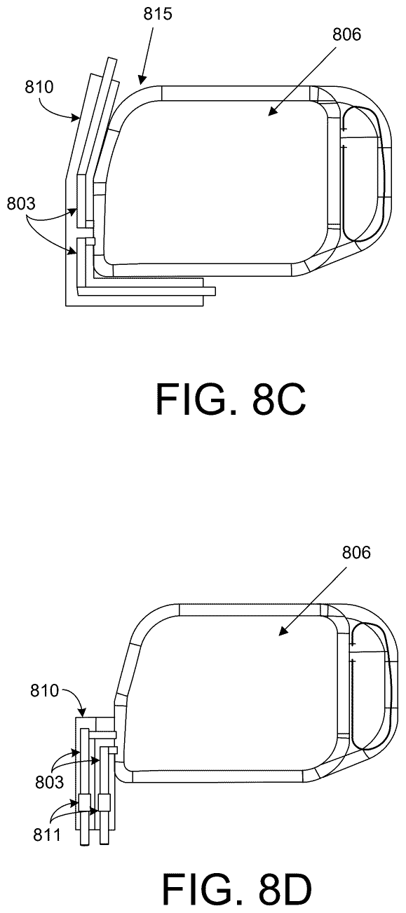

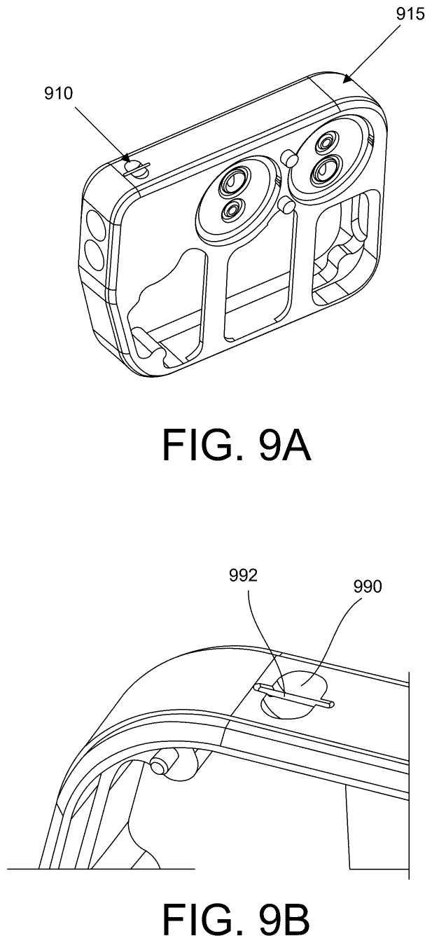

In some examples, the tube retainer 110 includes a clip member (e.g., see FIGS. 8A to 8D) that can be coupled to the manifold 115 of the pump device 106. The tube members 103, 105 can be snapped onto the clip member at various positions to route the tube members in the preferential location for a patient. In some examples, the tube retainer may include a post retainer (e.g., see FIGS. 9A and 9B) on an external surface of the manifold 115 of the pump device 106. In some examples, the post retainer includes a slot on the external surface and a post that extends across the slot.

FIGS. 2A to 2F illustrates an example of a retainer band 210 according to an aspect. The retainer band 210 includes one or more tabs defining a groove 222 (e.g., an open lumen) or a hole 223 (e.g., a closed lumen) configured to receive or hold a tube member 203 or 205 according to an aspect. The retainer band 210 is disposed around a pump device 206. The pump device 206 includes a manifold with a first fluid port 214 and a second fluid port 216. A tube member 203 may be coupled to (and extend from) from the first fluid port 214, and a tube member 205 may be coupled to (and extend from) from the second fluid port 216.

The retainer band 210 is an example of the tube retainer 110 of FIG. 1 and may include any of the details discussed with reference to that figure. In some examples, the retainer band 210 is an elastic band (e.g., including one or more clastic materials). In some examples, the retainer band 210 includes an elastic ring member. The retainer band 210 can be stretched (e.g., length and/or width can be stretched). In some examples, the retainer band 210 has a pre-defined shape that corresponds to a shape of a pump housing 226 (e.g., shown in FIG. 2B). In some examples, the retainer band 210 has a rectangular shape with round corner portions. In some examples, the retainer bands 210 are removable, where an operator may place one, two, or three retainer bands 210 (or more than three retainer bands 210) around the pump housing 226.

Referring to FIG. 2A, the retainer band 210 includes a side 224-1, a side 224-2, a side 224-3, and a side 224-4. Side 224-1 and side 224-2 are disposed opposite to each other (and parallel to each other), and side 224-3 and side 224-4 are disposed opposite to each other (and parallel to each other). In some examples, side 224-1 and side 224-2 are of equal length. In some examples, side 224-3 and side 224-4 are of equal length. In some examples, the length of sides 224-3, 224-4 are shorter than the length of sides 224-1, 224-2.

A retainer band 210 may include two or more tabs (e.g., tab 220-1, tab 220-2, tab 220-3, and/or tab 20-4) at different positions around the retainer band 210, where each tab defines a groove 222 (e.g., an open lumen) configured to hold a portion of a tube member 203 or 205. The size (e.g., diameter) of the groove 222 may be slightly less than the size (e.g., diameter) of the tube member 103 or 105. In some examples, the groove 222 extends in a direction A1. In some examples, the direction A1 is aligned with a length of the pump device 206. In some examples, the direction A1 is orthogonal to a length of the side 224-1 or the side 224-2. A direction A2 may define a height of the pump device 206, and may be perpendicular or orthogonal to the direction A1.

Referring to FIG. 2A, the retainer band 210 includes a tab 220-1 with a groove 222, and a tab 220-2 with a groove 222. The tab 220-1 is located on the side 224-1. The tab 220-2 is located on the side 224-2. In some examples, the tab 220-1 and the tab 220-2 are disposed opposite to each other. In some examples, the groove 222 is referred to as an open lumen. A tube member may be inserted into the groove 222. In some examples, a portion of a tube member 203 (as shown in FIG. 2D) may be inserted to the groove 222 of the tab 220-1, and a portion of a tube member 205 (as shown in FIG. 2D) may be inserted to the groove 222 of the tab 220-2.

As shown in FIG. 2B, multiple retainer bands 210 may be disposed around the pump device 206. For example, a retainer band 210-1 is disposed around the pump housing 226 at a first location in the direction A1, and a retainer band 210-2 is disposed around the pump housing 226 at a second location in the direction A1. The retainer band 210-1 includes a tab 220-1 with a groove 222 and a tab 220-2 with a groove 222. In some examples, the tab 220-1 of the retainer band 210-1 may be disposed on the side 224-3 of the retainer band 210-1, and the tab 220-2 of the retainer band 210-1 may be disposed on the side 224-4 of the retainer band 210-1. The tab 220-1 of the retainer band 210-1 may be disposed over the manifold 215 at a first location, and the tab 220-2 of the retainer band 210-1 may be disposed over the manifold 215 at a second location, where the first and second locations are opposite to each other. The retainer band 210-2 includes a tab 220-1 with a groove 222 and a tab 220-2 with a groove 222. In some examples, the tab 220-1 of the retainer band 210-2 may be disposed on the side 224-1 of the retainer band 210-2, and a tab (e.g., the tab 220-2) may be disposed on the side 224-2 of the retainer band 210-2. The tab 220-1 of the retainer band 210-2 may be disposed over a first side surface at a first location, and the tab 220-2 of the retainer band 210-2 may be disposed over a second side surface at a second location, where the first and second locations are opposite to each other. In some examples, referring to FIG. 2C, a retainer band 210-1, a retainer band 210-2, and a retainer band 210-3 may be disposed around the pump device 206.

In some examples, a separate tab with a groove 222 is disposed on each side of a retainer band 210. For example, referring to FIG. 2E, a retainer band 210 may include a tab 220-1 with a groove 222, a tab 220-2 with a groove 222, a tab 220-3 with a groove 222, and a tab 220-4 with a groove 222. The tab 220-1 may be disposed on the side 224-1, the tab 220-2 may be disposed on the side 224-2, the tab 220-3 may be disposed on the side 224-3, and the tab 220-4 may be disposed on the side 224-4. In some examples, the tab 220-1 and the tab 220-2 are disposed opposite to each other, and the tab 220-3 and the tab 220-4 are disposed opposite to each other.

Referring to FIG. 2F, in some examples, a tab 220 of a retainer band 210 may define a hole 223 (e.g., a closed lumen) configured to receive a tube member 203 or 205. In some examples, a retainer band 210 includes two tabs (each with a groove 222 or a hole 223) positioned on opposite band portions. For example, the retainer band 210 may have a rectangular shape with first and second (long) sides (e.g., side 224-1, side 224-2) and third and fourth (short) sides (e.g., side 224-3, side 224-4). In some examples, the two tabs are disposed on the first and second (long) sides (e.g., opposite to each other). In some examples, the two tabs are disposed on the third and fourth (short) sides (e.g., opposite to each other). In some examples, a retainer band 210 includes four tabs (e.g., one on each side, two tabs being disposed opposite to each).

FIG. 3A illustrates an example of a retainer tab 310-1 extending from an external surface of a manifold 315 at a first location, and a retainer tab 310-2 extending from an external surface of the manifold 315 at a second location. Each of the retainer tab 310-1 and the retainer tab 310-2 defines a groove 322 (e.g., an open lumen) configured to hold a portion of a tube member. FIG. 3B illustrates a detailed version of a retainer tab 310 with the groove 322 and suture holes 327-1, 327-2.

The manifold 315 includes a sidewall 330, a sidewall 332, a sidewall 334, and a sidewall 336. The manifold 315 includes a first fluid port 314 and a second fluid port 316. The first fluid port 314 and the second fluid port 316 are defined on the sidewall 330. A first tube member may be coupled to and extend from the first fluid port 314, and a second tube member may be coupled to and extend from the second fluid port 316. A portion of the first tube member may be inserted into the groove 322 of the retainer tab 310-1. A portion of the second tube member may be inserted into the groove 322 of the retainer tab 310-2. In some examples, the manifold 315 includes a valve 340 and a valve 342. In some examples, the outer surface of the sidewall 330, the sidewall 332, the sidewall 334, and the sidewall 336 form a portion of the outer surface of a pump device.

As shown in FIG. 3A, the retainer tab 310-1 may extend from the sidewall 332, and the retainer tab 310-2 may extend from the sidewall 336. In some examples, the retainer tab 310-1 and the retainer tab 310-2 are integral to the manifold 315. In some examples, the retainer tab 310-1 and the retainer tab 310-2 are constructed from the same material(s) as the manifold 315. In some examples, the retainer tab 310-1 and the retainer tab 310-2 are extensions of the manifold 315. As shown in FIG. 3B, a retainer tab 310 (e.g., either the retainer tab 310-1 or the retainer tab 310-2) includes a groove 322 and an opening 325 to the groove 322.

In some examples, the groove 322 and the opening 325 may be referred to as an open lumen. In some examples, the groove 322 includes a circular portion with a diameter. The groove 322 may extend in a direction A1. In some examples, a direction A2 is perpendicular or orthogonal to the direction A1. In some examples, a direction A3 is perpendicular or orthogonal to the direction A2. The opening 325 has a size that is less than the outer diameter of a tube member, which may require a press fit action to seat the tube member in the groove 322. In some examples, the retainer tab 310 includes a suture hole 327-1 and a suture hole 327-2. The suture holes 327-1, 327-2 may be used to attach sutures to the retainer tab 310. In some examples, the groove 322 is disposed between the suture hole 327-1 and the suture hole 327-2.

The retainer tab 310 includes an edge 341 coupled to the sidewall 332. The edge 341 may extend in the direction A3. The edge 341 may form a first end portion of the retainer tab 310. A second portion of the retainer tab 310 may be defined by an edge portion 347 and an edge portion 349, where the opening 325 is disposed between the edge portion 347 and the edge portion 349. The edge portion 347 and the edge portion 349 may extend in the direction A2. The retainer tab 310 includes a side edge 343 that extends from the edge 341 to the edge portion 347. The side edge 343 may extend in the direction A2. The retainer tab 310 includes a side edge 345 that extends from the edge 341 to the edge portion 349. The side edge 345 may extend in the direction A2. In some examples, the side edge 343 and the side edge 345 are tapered (e.g., the width in the direction A3 may become smaller as becoming closer to the opening 325). The body of the retainer tab 310 is defined by the edge 341, side edge 343, side edge 345, edge portion 347, and an edge portion 349, and a circular portion 321 defining the groove 322.

FIG. 4 illustrates a retainer hook 410 that extends from a side 436 of a manifold 415. A portion of a tube member may be inserted into and held by the retainer hook 410. In some examples, the retainer hook 410 includes a radius of curvature 428 that is smaller than the outer diameter of a tube member. In some examples, the retainer hook 410 includes a suture hole 427. In some examples, two retainer hooks 410 may be positioned on the manifold 415 in the same positions as shown in FIG. 3A.

FIG. 5A illustrates a retainer tab 510 according to another aspect. The retainer tab 510 defines a hole 523 (e.g., a closed lumen) configured to receive a tube member or a connector 511 (e.g., FIG. 5B) or a connector 513 (e.g., FIG. 5C). The retainer tab 510 is positioned on a manifold 515. The connector 511 or the connector 513 may be used to connect two tube members together. In some examples, the retainer tab 510 defines a suture hole 527-1 and a suture hole 527-2. In some examples, the hole 523 has a size (e.g., diameter) that is larger than a size (e.g., a diameter) of the suture hole 527-1 or the suture hole 527-2. In some examples, the hole 523 has a size (e.g., a diameter) that corresponds to (or slightly less) than the outer diameter of the tube member. In some examples, the hole 523 is disposed between the suture hole 527-1 and the suture hole 527-2.

The connector 511 includes a body 554, a cylindrical portion 552, and a cylindrical portion 556. The cylindrical portion 552 may be inserted into an end portion of a first tube member, and the cylindrical portion 556 may be inserted into an end portion of a second tube member. The body 554 may define one or more slots 558. The connector 513 may include a cylindrical portion 560, a circular disc portion 562, and a cylindrical portion 564. The cylindrical portion 560 may be inserted into an end portion of a first tube member, and the cylindrical portion 564 may be inserted into an end portion of a second tube member.

FIGS. 6A to 6D illustrate perspectives of a tube guide 610 coupled to a manifold 615 of a pump device 606 according to an aspect. In some examples, the tube guide 610 is referred to as a clip member. In some examples, the tube guide 610 has a C-shape. In some examples, the tube guide 610 is coupled to the fluid ports on the manifold 615 of the pump device 606. In some examples, the tube guide 610 is coupled to the pump device 606 using a mechanical lock (e.g., a snap fit), an adhesive bond, or applied as an over-mold.

The tube guide 610 includes an opening 674 configured to be coupled to (or aligned with) a first fluid port of the manifold 615, and an opening 676 configured to be coupled to (or aligned with) a second fluid port of the manifold 615. In some examples, the opening 674 is a through hole that extends through an exterior surface 671 and through an interior surface 673. In some examples, the opening 676 is a through hole that extends through an exterior surface 671 and through an interior surface 673. In some examples, as shown in FIG. 6D, the tube guide 610 includes a protrusion 678 (e.g., a circular protrusion, a circular lumen) that is inserted into the first fluid port, and a protrusion 680 (e.g., a circular protrusion, a circular lumen) that is inserted into the second fluid port. The protrusion 678 and the protrusion 680 extend from the interior surface 673.

The tube guide 610 defines two grooves, e.g., a first groove 670 (e.g., an open lumen) that extends from the opening 674, and a second groove 672 (e.g., an open lumen) that extends from the opening 676. In some examples, the first groove 670 and the second groove 672 are referred to as external channels, e.g., channels defined by the exterior surface 671. In some examples, the first groove 670 and the second groove 672 include circular grooves. In some examples, an opening to the groove 670 is slightly less than the outer diameter of a tube member. In some examples, an opening to the groove 672 is slightly less than the outer diameter of a tube member. First and second tube members (which extend from the first and second fluid ports, respectively) are positioned in the first and second grooves, 670, 672, respectively. The first and second grooves 670, 672 are defined by the exterior surface 671 of the tube guide 610. An end of the groove 670 includes an opening 682, and an end of the groove 672 includes an opening 684.

FIGS. 7A to 7D illustrate perspectives of a tube guide 710 coupled to a manifold 715 of a pump device 706 according to another aspect. In some examples, the tube guide 710 is referred to as a clip member. In some examples, the tube guide 710 has a C-shape. In some examples, the tube guide 710 is coupled to the fluid ports on the manifold 715 of the pump device 706. In some examples, the tube guide 710 is coupled to the pump device 706 using a mechanical lock (e.g., a snap fit), an adhesive bond, or applied as an over-mold.

The tube guide 710 includes an opening 774, on an interior surface 773, configured to be coupled to (or aligned with) a first fluid port of the manifold 715. The tube guide 710 includes an opening 776, on an interior surface 773, configured to be coupled to (or aligned with) a second fluid port of the manifold 715. In some examples, the opening 774 is an opening that extends through the interior surface 773 but not an exterior surface 771. In some examples, the opening 784 is an opening that extends through the interior surface 773 but not an exterior surface 771. In some examples, as shown in FIG. 7C, the tube guide 710 includes a protrusion 778 (e.g., a circular protrusion, a circular lumen) that is inserted into the first fluid port, and a protrusion 780 (e.g., a circular protrusion, a circular lumen) that is inserted into the second fluid port. The protrusion 778 and the protrusion 780 extend from the interior surface 673.

The tube guide 710 defines two internal grooves, e.g., a first groove 770 (e.g., a closed lumen) that extends from the opening 774, and a second groove 772 (e.g., a closed lumen) that extends from the opening 776. In some examples, the first groove 770 and the second groove 772 are referred to as internal channels, e.g., channels defined between the exterior surface 771 and the interior surface 773. In some examples, the first groove 770 and the second groove 772 include circular grooves. First and second tube members (which extend from the first and second fluid ports, respectively) are positioned in the first and second grooves, 770, 772, respectively. The first and second grooves 770, 772 are defined within the body of the tube guide 710. An end of the groove 770 includes an opening 782 (in which a tube member exits), and an end of the groove 772 includes an opening 784 (in which a tube member exits).

FIGS. 8A to 8D illustrate various aspects of a removable clip member 810 coupled to a manifold 815 of a pump device 806 according to an aspect. As shown in FIGS. 8A to 8D, the tube members 803 and/or connectors 811 can be snapped onto the clip member 810 at various positions to route the tube members 803 in the preferential location for a patient. FIGS. 9A and 9B illustrate perspectives of a post retainer 910 on an external surface of a manifold 915 of a pump device according to an aspect. In some examples, the post retainer 910 includes a slot 990 on the external surface and a post 992 that extends across the slot 990. In some examples, the post 992 and the slot 990 may be used to secure the device to tissue using a suture tie or other affixation member. For example, the affixation member (e.g., a suture tie) may be coupled (e.g., looped) through the post 992 to secure the device to tissue.

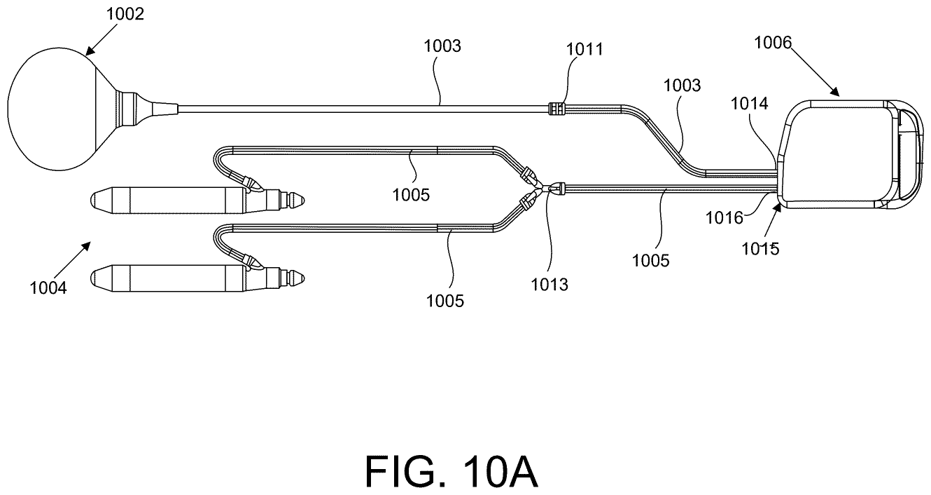

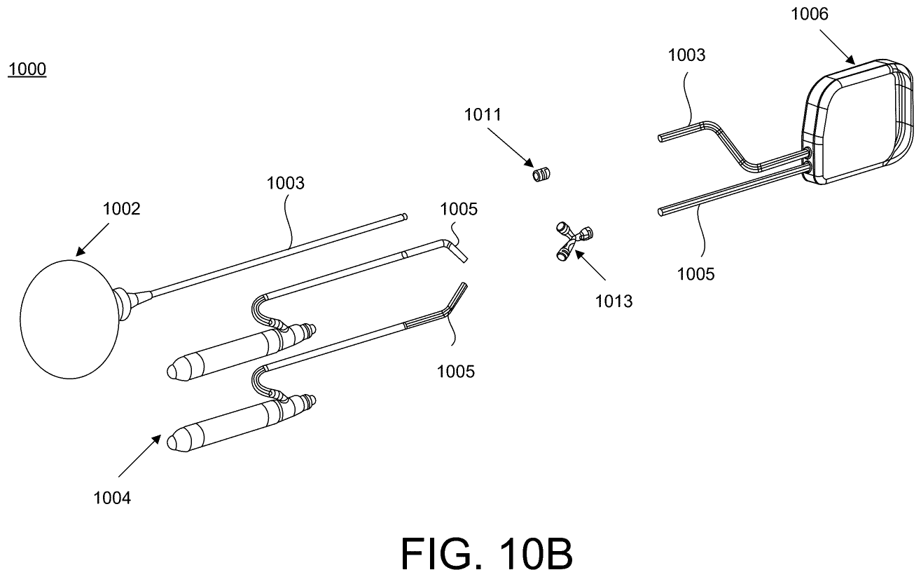

FIGS. 10A and 10B illustrate perspectives of an inflatable penile prosthesis 1000 according to an aspect. The inflatable penile prosthesis 1000 may be an example of any of the medical devices discussed herein, and, therefore, may include any of the details discussed with reference to the previous figures.

The inflatable penile prosthesis 1000 includes a pump device 1006. The pump device 1006 may be an example of any of the pump devices discussed with reference to the previous figures and may include any of the details discussed herein. The pump device 1006 includes fluidics components such as pumps, valves, sensing devices and the like positioned in fluid passageways. In some implementations, the pump device 1006 includes components such as, for example, one or more fluid control devices, one or more pressure sensors, and other such components. In some implementations, the pump device 1006 includes an electronic control system configured to provide for the transfer of fluid between a reservoir 1002 and an inflatable member 1004 via the fluidics components. In the example shown in FIGS. 10A and 10B, the inflatable member 1004 is in the form of a pair of inflatable cylinders. Fluidics components and the electronic components of the pump device 106 are included in a housing. In some examples, fluidics components and electronic components in the housing define a manifold 1015 (e.g., an electronically controlled fluid manifold) that provides for the electronic control of the flow of fluid between the reservoir 1002 and the inflatable member 1004.

The inflatable penile prosthesis 1000 includes one or more first tube members 1003 that connect a first fluid port 1014 of the manifold 1015 with the reservoir 1002. One or more second tube members 1005 connect a second fluid port 1016 of the manifold 1015 with the inflatable member 1004 in the form of the inflatable cylinders. In some examples, the inflatable penile prosthesis 1000 includes a connector 1011 that is used to connect two tube members 1003 together, and a connector 1013 that is used to connect two tube members 1005 together. In some examples, the pump device 1006 can communicate with an external controller, via respective communication modules. For example, an application stored in a memory and executed by a processor of the external controller may allow the user and/or a physician to operate, view, monitor and alter operation of the inflatable penile prosthesis 1000.

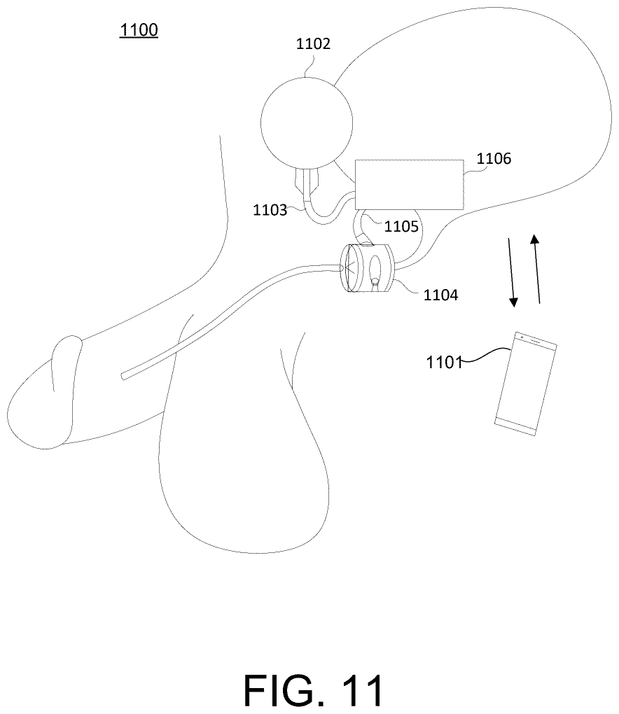

FIG. 11 illustrates a urinary control device 1100 having a pump device 1106 (e.g., an electronic pump assembly) according to an aspect. In some examples, the urinary control device 1100 is an artificial urinary sphincter device. The pump device 1106 may include any of the features of the pump devices discussed herein. The urinary control device 1100 includes a pump device 1106, a fluid reservoir 1102, and a cuff 1104 (e.g., an inflatable cuff).

The fluid reservoir 1102 may be a pressure-regulating inflation balloon or element. The fluid reservoir 1102 is in operative fluid communication with the cuff 1104 via one or more tube members 1103, 1105. The fluid reservoir 1102 is constructed of polymer material that is capable of elastic deformation to reduce fluid volume within the fluid reservoir 1102 and push fluid out of the fluid reservoir 1102 and into the cuff 1104. However, the material of the fluid reservoir 1102 can be biased or include a shape memory construct adapted to generally maintain the fluid reservoir 1102 in its expanded state with a relatively constant fluid volume and pressure. In some examples, this constant level of pressure exerted from the fluid reservoir 1102 to the cuff 1104 will keep the cuff 1104 at a desired inflated state when open fluid communication is provided between the fluid reservoir 1102 and the cuff 1104. In some examples, the fluid reservoir 1102 is implanted into the abdominal space.

A user may use an external device 1101 to control the urinary control device 1100. In some examples, the user may use the external device 1101 to inflate or deflate the cuff 1104. For example, in response to the user activating an inflation cycle using the external device 1101, the external device 1101 may transmit a wireless signal to the pump device 1106 to initiate the inflation cycle to transfer fluid from the fluid reservoir 1102 to the cuff 1104 (e.g., by opening an active valve where the pressure in the fluid reservoir 1102 causes the fluid to move through the active valve to the cuff 1104). In some examples, in response to the user activating a deflation cycle using the external device 1101, the external device 1101 may transmit a wireless signal to the pump device 1106 to initiate the deflation cycle to transfer fluid from the cuff 1104 to the fluid reservoir 1102.

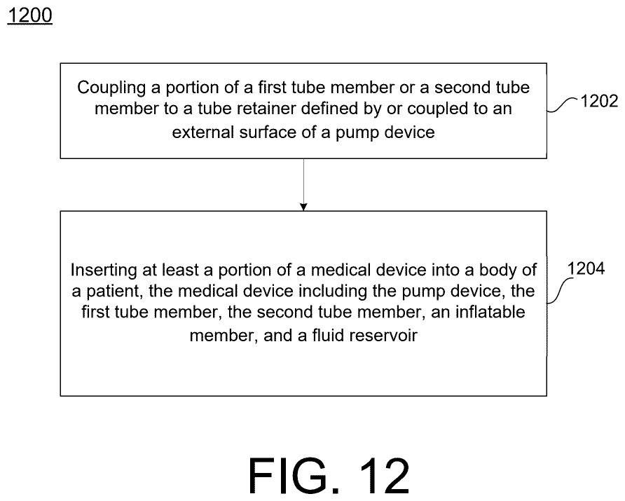

FIG. 12 illustrates a flowchart 1200 depicting example operations of coupling one or more tubing members to a tube retainer of a pump device according to an aspect. Although the flowchart 1200 of FIG. 12 illustrates the operations in sequential order, it will be appreciated that this is merely an example, and that additional or alternative operations may be included. Further, operations of FIG. 12 and related operations may be executed in a different order than that shown, or in a parallel or overlapping fashion.

Operation 1202 includes coupling a portion of a first tube member or a second tube member to a tube retainer defined by or coupled to an external surface of a pump device. Operation 1204 includes inserting at least a portion of a medical device into a body of a patient, the medical device including the pump device, the first tube member, the second tube member, an inflatable member, and a fluid reservoir.

Clause 1. An implantable medical device comprising: an inflatable member; a fluid reservoir; a pump device; a first tube member configured to fluidly connect the pump device and the fluid reservoir; a second tube member configured to fluidly connect the pump device and the inflatable member; and a tube retainer defined by or coupled to an external surface of the pump device, the tube retainer configured to hold a portion of the first tube member or the second tube member.

Clause 2. The implantable medical device of clause 1, wherein the tube retainer includes a removable retainer band, the removable retainer band configured to be disposed around a housing of the pump device, the removable retainer band including a first tab with a first groove configured to receive a portion of the first or second tube member, and a second tab with a second groove configured to receive a portion of the first or second tube member.

Clause 3. The implantable medical device of clause 2, wherein the removable retainer band including a third tab with a third groove configured to receive a portion of the first or second tube member, and a fourth tab with a fourth groove configured to receive a portion of the first or second tube member.

Clause 4. The implantable medical device of any one of clauses 1 to 3, wherein the pump device includes a manifold, the manifold including a first fluid port configured to be coupled to the first tube member, and a second fluid port configured to be coupled to the second tube member.

Clause 5. The implantable medical device of clause 4, wherein the tube retainer includes a first retainer tab extending from the manifold at a first location, the tube retainer including a second retainer tab extending from the manifold at a second location, the first retainer tab including a groove, a hole, or a hook configured to receive a portion of the first tube member, the second retainer tab including a groove, a hole, or a hook configured to receive a portion of the second tube member.

Clause 6. The implantable medical device of clause 5, wherein the first retainer tab includes one or more suture holes, and the second retainer tab includes one or more suture holes.

Clause 7. The implantable medical device of clause 4, wherein the tube retainer includes a tube guide coupled to the first fluid port and the second fluid port, the tube guide including a first opening and a first channel that extends from the first opening and along the tube guide in a first direction, the tube guide including a second opening and a second channel that extends from the second opening and along the tube guide in a second direction.

Clause 8. The implantable medical device of clause 7, wherein the first channel is configured to receive a portion of the first tube member, and the second channel is configured to receive a portion of the second tube member.

Clause 9. The implantable medical device of clause 7, wherein the first channel and the second channel are external channels defined by an external surface of the tube guide.

Clause 10. The implantable medical device of clause 7, wherein the first channel and the second channel are internal channels defined within a body of the tube guide.

Clause 11. The implantable medical device of any one of clauses 1 to 10, wherein the tube retainer includes a clip member configured to be coupled to the pump device, the first and second tube members configured to be clipped to the clip member.

Clause 12. The implantable medical device of clause 4, wherein the tube retainer includes a post retainer defined on the manifold, the post retainer includes a slot in an external surface of the manifold, and a post disposed over the slot.

Clause 13. A method comprising: coupling a portion of a first tube member or a second tube member to a tube retainer defined by or coupled to an external surface of a pump device; and inserting at least a portion of a medical device into a body of a patient, the medical device including the pump device, the first tube member, the second tube member, an inflatable member, and a fluid reservoir.

Clause 14. The method of clause 13, wherein the tube retainer includes a retainer band, the method comprising: placing the retainer band around a housing of the pump device, the retainer band including a first tab with a first groove, and a second tab with a second groove; inserting a first portion of the first tube member or the second tube member into the first groove; and inserting a second portion of the first tube member or the second tube member into the second groove.

Clause 15. The method of clause 13 or 14, wherein the pump device includes a manifold, the tube retainer including a first retainer tab extending from the manifold at a first location, the tube retainer including a second retainer tab extending from the manifold at a second location, the first retainer tab including a groove, a hole, or a hook, the second retainer tab including a groove, a hole, or a hook, the method comprising: inserting a portion of the first tube member into the groove, the hole, or the hook of the first retainer tab; and inserting a portion of the second tube member into the groove, the hole, or the hook of the second retainer tab.

Clause 16. An implantable medical device comprising: an inflatable member; a fluid reservoir; a pump device; a first tube member configured to fluidly connect the pump device and the fluid reservoir; a second tube member configured to fluidly connect the pump device and the inflatable member; and a tube retainer defined by or coupled to an external surface of the pump device, the tube retainer configured to hold a portion of the first tube member or the second tube member.

Clause 17. The implantable medical device of clause 16, wherein the tube retainer includes a removable retainer band, the removable retainer band configured to be disposed around a housing of the pump device, the removable retainer band including a first tab with a first groove configured to receive a portion of the first or second tube member, and a second tab with a second groove configured to receive a portion of the first or second tube member.

Clause 18. The implantable medical device of clause 17, wherein the removable retainer band including a third tab with a third groove configured to receive a portion of the first or second tube member, and a fourth tab with a fourth groove configured to receive a portion of the first or second tube member.

Clause 19. The implantable medical device of clause 16, wherein the pump device includes a manifold, the manifold including a first fluid port configured to be coupled to the first tube member, and a second fluid port configured to be coupled to the second tube member.

Clause 20. The implantable medical device of clause 19, wherein the tube retainer includes a first retainer tab extending from the manifold at a first location, the tube retainer including a second retainer tab extending from the manifold at a second location, the first retainer tab including a groove, a hole, or a hook configured to receive a portion of the first tube member, the second retainer tab including a groove, a hole, or a hook configured to receive a portion of the second tube member.

Clause 21. The implantable medical device of clause 20, wherein the first retainer tab includes one or more suture holes, and the second retainer tab includes one or more suture holes.

Clause 22. The implantable medical device of clause 19, wherein the tube retainer includes a tube guide coupled to the first fluid port and the second fluid port, the tube guide including a first opening and a first channel that extends from the first opening and along the tube guide in a first direction, the tube guide including a second opening and a second channel that extends from the second opening and along the tube guide in a second direction.

Clause 23. The implantable medical device of clause 22, wherein the first channel is configured to receive a portion of the first tube member, and the second channel is configured to receive a portion of the second tube member.

Clause 24. The implantable medical device of clause 22, wherein the first channel and the second channel are external channels defined by an external surface of the tube guide.

Clause 25. The implantable medical device of clause 22, wherein the first channel and the second channel are internal channels defined within a body of the tube guide.

Clause 26. The implantable medical device of clause 16, wherein the tube retainer includes a clip member configured to be coupled to the pump device, the first and second tube members configured to be clipped to the clip member.

Clause 27. The implantable medical device of clause 19, wherein the tube retainer includes a post retainer defined on the manifold, the post retainer includes a slot in an external surface of the manifold, and a post disposed over the slot.

Clause 28. A method comprising: coupling a portion of a first tube member or a second tube member to a tube retainer defined by or coupled to an external surface of a pump device; and inserting at least a portion of a medical device into a body of a patient, the medical device including the pump device, the first tube member, the second tube member, an inflatable member, and a fluid reservoir.

Clause 29. The method of clause 28, wherein the tube retainer includes a retainer band, the method comprising: placing the retainer band around a housing of the pump device, the retainer band including a first tab with a first groove, and a second tab with a second groove; inserting a first portion of the first tube member or the second tube member into the first groove; and inserting a second portion of the first tube member or the second tube member into the second groove.

Clause 30. The method of clause 28, wherein the pump device includes a manifold, the tube retainer including a first retainer tab extending from the manifold at a first location, the tube retainer including a second retainer tab extending from the manifold at a second location, the first retainer tab including a groove, a hole, or a hook, the second retainer tab including a groove, a hole, or a hook, the method comprising: inserting a portion of the first tube member into the groove, the hole, or the hook of the first retainer tab; and inserting a portion of the second tube member into the groove, the hole, or the hook of the second retainer tab.

Clause 31. An implantable medical device comprising: an inflatable member; a fluid reservoir; a pump device including a manifold, the manifold including a first fluid port and a second fluid port; a first tube member configured to connect to the first fluid port and to the fluid reservoir; a second tube member configured to fluidly connect to the second fluid port and the inflatable member; and a tube retainer defined by or coupled to an external surface of the pump device, the tube retainer configured to hold a portion of the first tube member or the second tube member.

Clause 32. The implantable medical device of clause 31, wherein the tube retainer includes a removable retainer band, the removable retainer band configured to be disposed around a housing of the pump device, the removable retainer band including a first tab with a first groove configured to receive a portion of the first or second tube member, and a second tab with a second groove configured to receive a portion of the first or second tube member, the removable retainer band including a third tab with a third groove configured to receive a portion of the first or second tube member, and a fourth tab with a fourth groove configured to receive a portion of the first or second tube member.

Clause 33. The implantable medical device of clause 31, wherein the tube retainer includes a first retainer tab extending from the manifold at a first location, the tube retainer including a second retainer tab extending from the manifold at a second location, the first retainer tab including a groove, a hole, or a hook configured to receive a portion of the first tube member, the second retainer tab including a groove, a hole, or a hook configured to receive a portion of the second tube member, wherein the first retainer tab includes one or more suture holes, and the second retainer tab includes one or more suture holes.

Clause 34. The implantable medical device of clause 31, wherein the tube retainer includes a tube guide coupled to the first fluid port and the second fluid port, the tube guide including a first opening and a first channel that extends from the first opening and along the tube guide in a first direction, the tube guide including a second opening and a second channel that extends from the second opening and along the tube guide in a second direction, wherein the first channel is configured to receive a portion of the first tube member, and the second channel is configured to receive a portion of the second tube member.

Clause 35. The implantable medical device of clause 34, wherein the first channel and the second channel are external channels defined by an external surface of the tube guide.

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implantable medical device comprising:
an inflatable member;
a fluid reservoir;
a pump device;
a first tube member configured to fluidly connect the pump device and the fluid reservoir;
a second tube member configured to fluidly connect the pump device and the inflatable member; and
a tube retainer defined by or coupled to an external surface of the pump device, the tube retainer configured to hold a portion of the first tube member or the second tube member.

2. The implantable medical device of claim 1, wherein the tube retainer includes a removable retainer band, the removable retainer band configured to be disposed around a housing of the pump device, the removable retainer band including a first tab with a first groove configured to receive a portion of the first or second tube member, and a second tab with a second groove configured to receive a portion of the first or second tube member.

3. The implantable medical device of claim 2, wherein the removable retainer band including a third tab with a third groove configured to receive a portion of the first or second tube member, and a fourth tab with a fourth groove configured to receive a portion of the first or second tube member.

4. The implantable medical device of claim 1, wherein the pump device includes a manifold, the manifold including a first fluid port configured to be coupled to the first tube member, and a second fluid port configured to be coupled to the second tube member.

5. The implantable medical device of claim 4, wherein the tube retainer includes a first retainer tab extending from the manifold at a first location, the tube retainer including a second retainer tab extending from the manifold at a second location, the first retainer tab including a groove, a hole, or a hook configured to receive a portion of the first tube member, the second retainer tab including a groove, a hole, or a hook configured to receive a portion of the second tube member.

6. The implantable medical device of claim 5, wherein the first retainer tab includes one or more suture holes, and the second retainer tab includes one or more suture holes.

7. The implantable medical device of claim 4, wherein the tube retainer includes a tube guide coupled to the first fluid port and the second fluid port, the tube guide including a first opening and a first channel that extends from the first opening and along the tube guide in a first direction, the tube guide including a second opening and a second channel that extends from the second opening and along the tube guide in a second direction.

8. The implantable medical device of claim 7, wherein the first channel is configured to receive a portion of the first tube member, and the second channel is configured to receive a portion of the second tube member.

9. The implantable medical device of claim 7, wherein the first channel and the second channel are external channels defined by an external surface of the tube guide.

10. The implantable medical device of claim 7, wherein the first channel and the second channel are internal channels defined within a body of the tube guide.

11. The implantable medical device of claim 1, wherein the tube retainer includes a clip member configured to be coupled to the pump device, the first and second tube members configured to be clipped to the clip member.

12. The implantable medical device of claim 4, wherein the tube retainer includes a post retainer defined on the manifold, the post retainer includes a slot in an external surface of the manifold, and a post disposed over the slot.

13. A method comprising:
coupling a portion of a first tube member or a second tube member to a tube retainer defined by or coupled to an external surface of a pump device; and
inserting at least a portion of a medical device into a body of a patient, the medical device including the pump device, the first tube member, the second tube member, an inflatable member, and a fluid reservoir.

14. The method of claim 13, wherein the tube retainer includes a retainer band, the method comprising:
placing the retainer band around a housing of the pump device, the retainer band including a first tab with a first groove, and a second tab with a second groove;
inserting a first portion of the first tube member or the second tube member into the first groove; and
inserting a second portion of the first tube member or the second tube member into the second groove.

15. The method of claim 13, wherein the pump device includes a manifold, the tube retainer including a first retainer tab extending from the manifold at a first location, the tube retainer including a second retainer tab extending from the manifold at a second location, the first retainer tab including a groove, a hole, or a hook, the second retainer tab including a groove, a hole, or a hook, the method comprising:
inserting a portion of the first tube member into the groove, the hole, or the hook of the first retainer tab; and
inserting a portion of the second tube member into the groove, the hole, or the hook of the second retainer tab.

16. An implantable medical device comprising:
an inflatable member;
a fluid reservoir;
a pump device including a manifold, the manifold including a first fluid port and a second fluid port;
a first tube member configured to connect to the first fluid port and to the fluid reservoir;
a second tube member configured to fluidly connect to the second fluid port and the inflatable member; and
a tube retainer defined by or coupled to an external surface of the pump device, the tube retainer configured to hold a portion of the first tube member or the second tube member.

17. The implantable medical device of claim 16, wherein the tube retainer includes a removable retainer band, the removable retainer band configured to be disposed around a housing of the pump device, the removable retainer band including a first tab with a first groove configured to receive a portion of the first or second tube member, and a second tab with a second groove configured to receive a portion of the first or second tube member, the removable retainer band including a third tab with a third groove configured to receive a portion of the first or second tube member, and a fourth tab with a fourth groove configured to receive a portion of the first or second tube member.

18. The implantable medical device of claim 16, wherein the tube retainer includes a first retainer tab extending from the manifold at a first location, the tube retainer including a second retainer tab extending from the manifold at a second location, the first retainer tab including a groove, a hole, or a hook configured to receive a portion of the first tube member, the second retainer tab including a groove, a hole, or a hook configured to receive a portion of the second tube member, wherein the first retainer tab includes one or more suture holes, and the second retainer tab includes one or more suture holes.

19. The implantable medical device of claim 16, wherein the tube retainer includes a tube guide coupled to the first fluid port and the second fluid port, the tube guide including a first opening and a first channel that extends from the first opening and along the tube guide in a first direction, the tube guide including a second opening and a second channel that extends from the second opening and along the tube guide in a second direction, wherein the first channel is configured to receive a portion of the first tube member, and the second channel is configured to receive a portion of the second tube member.

20. The implantable medical device of claim 19, wherein the first channel and the second channel are external channels defined by an external surface of the tube guide.
